(19) United States
(12) Patent Application Publication
(10) Pub. No.: US 2006/0288942 A1
(43) Pub. Date: Dec. 28, 2006

(54) Modular apparatus for harvesting cultured nematodes and uses thereof

(76) Inventor: James P. Miller, Streamwood, IL (US)

Correspondence Address: David G. Rosenbaum, Esq.;Rosenbaum & Associates, P.C., Suite #380, 650 Dundee Road, Northbrook, IL, 60062, US

(21) Appl. No.: 11/158,501

(22) Filed: Jun. 22, 2005

(51) Int. Cl. A01K 67/033

(52) U.S. Cl. 119/6.7

(57) ABSTRACT

The present invention provides a harvesting system for obtaining large quantities of nematodes that are minimally contaminated with growth medium, as well as methods for using this system. One feature of the present invention provides a harvesting system for nematodes in which culture medium contamination of the harvested nematodes is minimized. A second feature of the present invention provides a harvesting system for nematodes in which the number of nematodes recovered in a single harvest is maximized.

### BACKGROUND OF THE INVENTION

Nematodes are a large group of generally microscopic roundworms with diverse feeding habits. Specifically, there are more than 500,000 recognized species of nematodes, with these animals generally between 400 μm and 5 mm long, although some members of the group attain lengths of greater than one foot. Although some species of nematode parasitize animals or plants, most nematodes feed on bacteria, fungi, or other soil organisms.

Because of their ubiquity and range of diet, nematodes have been used in a variety of commercial applications, particularly in pest control. One of the best known examples of such an application is the use of insectivorous “beneficial nematodes” in agricultural applications to control, e.g., root weevils, and other insects. In addition, beneficial nematodes also control other insect pests, for example flea larvae, thus providing an additional rationale for such nematode application.

In addition to their use in pest control, nematodes also find use as a food source, particularly in industrial aquaculture, i.e., large-scale fish production. Specifically, live foods are required by the larvae (young) of many commercially important fish, with prepared artificial diets either not eaten by these larvae or, if eaten, either not assimilated or an inadequate nutritional source for proper growth rates during critical developmental stages. Although brine shrimp (Artemia sp.) are routinely used as a larval food source, there are disadvantages to the use of Artemia, including their inconsistent nutritional quality, their size, their consumption of larval food, and their price. Thus increased attention has focused on the use of nematodes as an alternative food source in commercial fish rearing.

One nematode species of particular interest in aquaculture is the free-living nematode species Panagrellus redivivus. Nematodes of this species have several advantages for such applications, including: their ease of culturing; their size, which is more appropriate for smaller fish larvae than, e.g., Artemia; and, their high nutritional content. Thus a variety of studies have examined the use of such nematodes in aquaculture, as well as methods for industrial-scale production of these organisms, e.g., large-volume bioreactors and the like.

The advantages of nematodes as feed for fish apply equally to small-scale aquaculture operations as they do for production on a commercial scale; however, different methods are required for the small-scale production of nematodes than those used for a commercial operation. In this regard, of particular concern are methods for removing contaminating culture medium from harvested nematodes as well as harvest yield. Specifically, while harvesting and purification of harvested nematodes are done in commercial systems via, e.g., filtration and sequential washings, such methods are not feasible for a small-scale operation in which apparatus costs are limiting, and plumbing requirements are usually inadequate to such bulk purification methods. Also in general contrast to commercial operations, in small-scale systems nematode cultures are usually maintained for relatively long periods with only partial harvesting as needed for feed production. Thus commercial methods that involve harvesting an entire culture (i.e., “complete” harvests) are generally unsuitable to such small-scale, partial-harvest (synonymously, “continuous harvest”) operations.

To date, there have been only a limited number of systems developed for harvesting nematode populations for small-scale aquaculture applications, and all of these systems suffer from problems with contamination of harvested populations by growth medium and scale of recovery of nematodes. Specifically, to date methods for small-scale nematode recovery generally rely on the motility of nematodes on a single surface of relatively small area, e.g., a spatula, wooden tongue depressor, etc., either placed on the semi-solid nematode culture medium or inserted vertically or at an incline into this semi-solid medium. Thus for example a flat harvesting surface such as a wooden tongue depressor is inserted into a semi-solid cake of oatmeal-based medium on which nematodes are cultured, and, after a suitable time delay during which nematodes ascend onto the harvesting surface via, e.g., sinusoidal body movements, the harvesting surface is withdrawn and immersed into a fish tank where the nematodes are quickly dispersed and consumed. Although such systems are inexpensive and moderately effective, they are limited both in terms of the contamination of the withdrawn nematodes by culture medium adhering to the harvesting surface and in the number of nematodes that can be withdrawn on the harvesting surface.

There is thus a need for a small-scale nematode harvesting system for obtaining nematodes minimally contaminated with growth medium. There is additionally a need for such systems where the number of nematodes obtained by harvesting is maximized.

### SUMMARY OF THE INVENTION

The present invention provides a harvesting system for obtaining large quantities of nematodes that are minimally contaminated with growth medium, as well as methods for using this system. One feature of the present invention provides a harvesting system for nematodes in which culture medium contamination of the harvested nematodes is minimized. A second feature of the present invention provides a harvesting system for nematodes in which the number of nematodes recovered in a single harvest is maximized.

Thus the present invention can be used to produce pure cultures of large numbers of nematodes. One aspect of the present invention particularly contemplates the use of these cultures in aquaculture operations, including both fish feeding applications and in order to provide nematodes for research and development efforts for improved aquaculture methods. In these applications Panagrellus redivivus is preferred. However, the present invention is not intended to be limited to aquaculture applications only, or to Panagrellus redivivus only. Instead, the present invention may be used for any application in which pure cultures of relatively large numbers of nematodes are required, including ones involving the effects of parasitic fungi on beneficial nematodes, the effects of environmental challenges on nematodes (i.e., nematodes as a barometer for environmental health), the use of nematodes in genetic and longevity studies, etc.

One embodiment of the present invention is directed to an apparatus for harvesting nematodes, where said apparatus comprises more than one collection plate member in order to provide a large surface area for nematode harvest, thereby increasing the yield of nematodes obtained in any one harvest. In this embodiment, the collection plate members may be in direct contact with the culture medium, or they may be suspended above the medium.

Another embodiment of the present invention is directed to an apparatus for harvesting nematodes, wherein said apparatus comprises at least one paired set of collection plate member and corresponding base member, wherein said collection plate member is in removable contact with said base member. In one aspect, this embodiment comprises a single paired set of collection member and corresponding base member; in another embodiment this harvesting apparatus comprises more than one paired set of collection plate member and corresponding base member. The collection plate member and base member of the present invention are embodied by a variety of materials, including glass, plastic, metal, ceramic, and wood. In addition, these materials may be coated for suitability for nematodes.

Another embodiment of the present invention is directed to a method for harvesting nematodes, wherein said method comprises: contacting the base member of the paired set of collection plate member and base member of the apparatus with nematode culture medium; allowing nematodes to collect on the collection plate member of the paired set of collection plate member and base member of the apparatus; and, removing the collection plate member of the paired set of collection plate member and base member of the apparatus for nematode harvesting.

### BRIEF DESCRIPTION OF THE DRAWINGS

The foregoing summary as well as the following detailed description of preferred embodiments of the present invention will be better understood when read in conjunction with the appended drawings. For the purpose of illustrating the present invention, there are shown in the drawings embodiments which are presently preferred. It should be understood, however, that the present invention is not limited to the precise arrangements and instrumentalities shown.

FIG. 1A provides a front elevation view of a base member of an embodiment of the present invention.

FIG. 1B provides a front elevation view of a spacer of an embodiment of the present invention.

FIG. 1C provides a perspective view of a combination of base member and two spacers of an embodiment of the present invention.

Figure 1D provides a front elevation view of a collection plate member of an embodiment of the present invention.

FIG. 2 (lower portion) provides a perspective view of a plurality of base members and associated spacers of an embodiment of the present invention.

FIG. 2 (upper portion) provides a perspective view of a plurality of collection plate members of an embodiment of the present invention.

FIG. 3 provides a perspective view of a plurality of collection plate members of an embodiment of the present invention contacted with a plurality of base members and associated spacers of an embodiment of the present invention.

### DETAILED DESCRIPTION OF THE INVENTION

The present invention provides a harvesting system for obtaining large quantities of nematodes that are minimally contaminated with growth medium, as well as methods for using this system. In one aspect, the present invention is directed to an apparatus comprising more than one collection plate member in order to provide a large surface area for nematode harvest, thereby increasing the yield of nematodes obtained in any one harvest. In a preferred aspect for increasing both yield and minimizing contamination by medium, the harvesting system of the invention is based on the use of an apparatus comprising at least one paired set of collection plate member and corresponding base member, where the collection plate member is in removable contact with the base member. In use, the base member contacts the semi-solid culture medium (e.g., rests on the surface of the culture medium or is partially embedded in the surface of the culture medium) such that nematodes are able to migrate to the base member (by, e.g., active motion as well as by diffusion); once on the base member, nematodes then continue to migrate to the collection plate member, which is subsequently removed from the base member for harvesting of nematodes.

One aim of the present invention is to provide a harvesting system for nematodes in which culture medium contamination of the harvested nematodes is minimized. In the harvesting system of the present invention, the base member plays an important part in realizing this goal. Specifically, in addition to serving as a pathway for migration of nematodes from the semi-solid culture medium to the collection plate member, the base member acts as a separator between the culture medium and the collection plate member, thereby ensuring that there is minimal contamination of the collection plate member by the culture medium.

A second aim of the present invention is to provide a harvesting system for nematodes in which the number of nematodes recovered in a single harvest is maximized., i.e., where a large “harvest yield” or “nematode yield” is obtained. In the harvesting system of the present invention, this goal is provided in a preferred embodiment comprising a plurality of paired sets of collection plate member and corresponding base member, i.e., a preferred embodiment in which multiple collection plates are used to obtain a large surface area for nematode harvest.

Thus in one aspect, the present invention is directed to an apparatus comprising more than one collection plate member in order to provide a large surface area for nematode harvest, thereby increasing the yield of nematodes obtained in any one harvest. In a preferred aspect for increasing both yield and minimizing contamination by medium, the present invention is directed to an apparatus comprising at least one paired set of collection plate member and corresponding base member, where the collection plate member is in removable contact with the base member. “Collection plate member” as contemplated herein refers to a generally flat member onto which nematodes migrate from the semi-solid culture medium via the base member. Thus the collection plate members of the present invention may be of any material suitable for nematodes, including, but not limited to, relatively solid-surfaced materials such as glass, plastic, metal, wood, etc., as well as porous or mesh-like materials, such as ceramic, open-weave materials etc. The choice of a particular material for the collection plate member will be made based on cost, longevity, suitability for nematodes, etc. Thus for example one preferred embodiment of the present invention is directed to polycarbonate or acrylic collection plate members; however, for some applications it may be advantageous to use a mesh-like material capable of providing a greater effective surface area for nematode collection.

In addition to the use of different materials for the collection plate members of the present invention, also contemplated herein are different coatings for such materials. Plastics, for example, are often modified by one or more coatings when they are used in biological applications such as tissue culture. Thus the present invention includes the use of various such coatings for plastics (or combination of coatings), or other coatings (or combination of coatings) as are suitable for nematodes.

“Suitability for nematodes,” as used herein, refers to a material or material coating for the collection plate member or base member of the present invention that do not cause: the death of nematodes; the failure of nematodes to migrate to the collection plate member; the failure of nematodes to adhere to the collection plate member, etc. Particularly suitable materials will be those materials that have advantageous wetting and/or nematode-adhesive properties. Suitability may be readily assayed by measuring the number of nematodes collected from a collection plate member of the harvesting system of the present invention as a function of nematode population in the culture medium, with no nematodes harvested indicative of a material or coating that is “unsuitable.” By the same criteria, the greater the number of nematodes harvested from the collection plate member, the more suitable the material or coating (or both) for the apparatus of the present invention.

As used herein, “base member” refers to the portion of the apparatus in removable contact with the collection plate member. Materials used for the base member are selected from the same materials as those used for the collection plate member, using similar criteria. In this regard, however, there may be some situations in which it is advantageous to have collection plate members with a higher affinity for nematodes than is the case for the corresponding base members. Specifically, it may be advantageous to have base members which are optimized for motility of nematodes from culture medium to collection plate member, rather than for retention, as would be preferred in some instances for the collection plate members of the harvesting apparatus of the present invention. As for the collection plate member, the material used for fabrication of the base member may be coated or uncoated.

The base members of the present invention may be solid; also contemplated are base members that have been modified to allow for passage of nematodes through them, e.g., by providing openings in the base member to allow for such passage. Such openings may be formed in a variety of ways, for example by drilling the base members, by forming the casting the base members on molds such that these openings are pre-formed, etc. The choice of opening configuration is contemplated to include any configuration that comports with the goals of the present invention of increasing nematode yield, improving culture longevity, etc.

Although the base member may be of any appropriate configuration, as for the collection plate member, a generally flat base member is preferred. In this regard, three criteria are particularly relevant in selecting the shape of the base member: the extent of contact boundary between the base member and the collection plate member; the extent of contact boundary between the base member and the semi-solid medium; and, the surface area of the semi-solid medium occluded by the base member, i.e., the “footprint” area of the base member blocking the surface of the semi-solid medium. As used herein, “contact boundary” refers to the length of contact between the surfaces of the collection plate member and base member so that, for example, if both collection plate member and base member had a rectangular cross-section of 2 mm×12 mm, the contact boundary would be the circumference of either rectangle, i.e., 28 mm.

With regard to the extent of contact boundary between the base member and the collection plate member, it is preferable that this contact boundary be large such that there is a large region available for nematodes moving from the base member to the collection plate member. For similar functional reasons, it is also preferable that the extent of contact boundary between the base member and the semi-solid medium be large. Therefore, although a preferred embodiment of the present invention contemplates flat collection plates and contacting base members, in order to increase contact area between the collection plates and corresponding base members, both collection plates and base members may have non-flat faces, e.g., have undulating, rippled, zig-zagged faces, etc. In addition to increasing the contact area between collection plates and base members, such non-flat surfaces will also increase the surface area of the collection plates, thereby potentially increasing nematode yield. Additionally, such non-flat surfaces may also increase nematode yield by providing protected pockets for nematode accumulation, etc. The choice of a particular face configuration will be made empirically based on assays for nematode recovery from the collection plates, i.e., nematode yield.

With regard to footprint area, it is important there not be too large a footprint area of the base member on the semi-solid medium. Specifically, a large footprint area impedes access of nematodes to culture medium for feeding and motion, decreases liquid and gaseous circulation between the surface of the media and the surrounding air/water environment, and may increase the possibility of bacterial or other contaminant growth. Thus, for example, the present invention contemplates a base member which covers no more than about 10, 15, 20, 25, 30, 35, or 40% of the surface of the culture medium.

In the present invention, every collection plate member is paired with a corresponding base member, with the collection plate member in removable contact with the base member. Thus each collection plate member is positioned so as to contact a corresponding base member (which itself contacts the culture medium), with the collection plate member removable from contact with the base member for harvesting.

“Removable contact” as used herein is intended to encompass any method of removably contacting the collection plate member with its corresponding base member; thus, although a preferred embodiment of a mechanical design providing such functionality is provided in the Figures, it should be understood that other feasible designs are also envisioned in the present invention. For example, in addition to the embodiment presented in the Figures the present invention contemplates, e.g., a mechanism for providing removable contact comprising two protruding tabs of material in the base-member-facing face of the collection plate member which insert into two corresponding recesses in the collection-plate-member-facing face of the base member. Other arrangements capable of producing a removable contact between collection plate member and corresponding base member would be well within the knowledge of the skilled artisan, when provided with the disclosure of the present invention.

In the present invention, the base member serves to minimize contamination by culture medium of the collection plate member, thereby minimizing contamination by culture medium of collected nematodes. Although in some situations a single pair of collection plate member and corresponding base member may be used, a plurality of such paired sets of collection plate member and base member are preferred. Specifically, a plurality of collection plate members provide a greater surface area for nematode harvest, i.e., allow for more nematodes to be collected with each removal of the plurality of collection plate members from the corresponding base members. Thus in a modular design for arranging a plurality of collection plate members and corresponding base members such as that presented in the Figures and described below, more paired collection plate member/base member sets may be easily added to increase nematode yield.

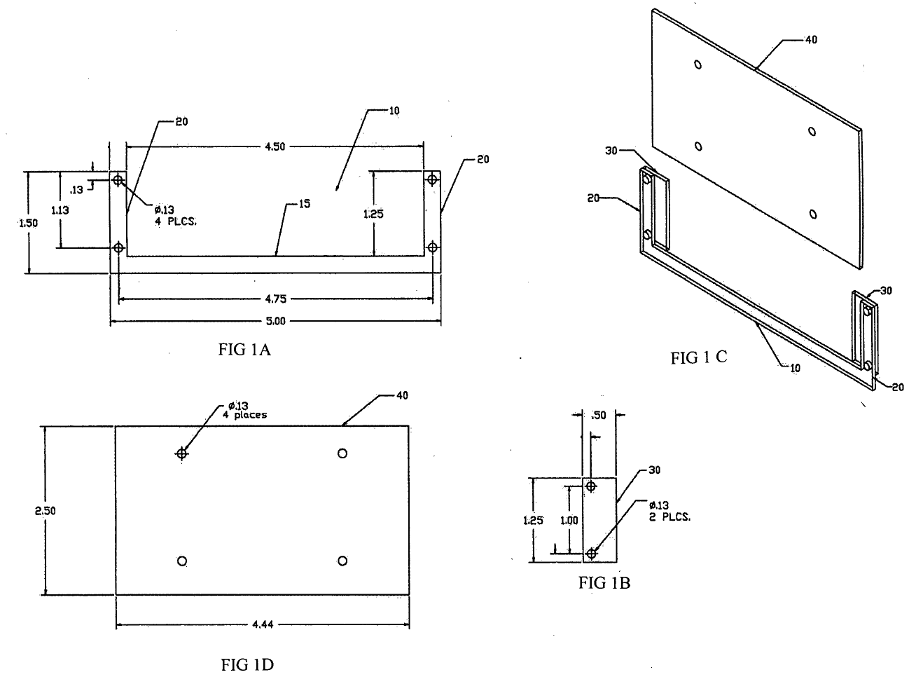

When there are more than one pair of collection plate member and corresponding base member, it is contemplated that these multiple pairs will be joined to one another so as to maintain a space between each pair so as to allow both sides of the collection plate member and corresponding base member to be available for access by nematodes. Although the Figures provide one embodiment of the present invention in which a separate spacer element is used to separate each collection plate member/base member pair (see, e.g., FIG. 1B), any mechanical design providing such spacing is contemplated herein.

As discussed above, the base member or members of the present invention are placed in contact with (i.e., on the surface of, or partially embedded within) the culture medium of the present invention. Although the Figures show the plurality of base members of this embodiment oriented perpendicular to the surface of the culture medium, situations in which these base members are oriented at an angle relative to the surface of the culture medium are also explicitly contemplated in the present invention. Similarly, although the Figures show an embodiment of the invention in which there are a plurality of collection plate members, all of which are parallel to the plurality of base members of this embodiment, situations in which the collection plate members (or member, when there is only a single pair of collection plate member and corresponding base member) is tilted at an angle relative to the base members are also contemplated.

As used herein, the terms “culture medium” or “semi-solid” medium refer to any of the variety of media used to grow Panagrellus redivivus, e.g., oatmeal-based pastes and the like. A specific example of culture media is given in, for example, Example 1.

In use, the combination of the apparatus of the invention in contact with the appropriate culture medium must be housed in a suitable container. The selection of a container is made based on such criteria as the size of the harvesting apparatus of the present invention, the volume of the culture medium to be used, the ability of the container to be covered in such a way as to protect the apparatus/culture medium combination from airborne contaminants or other materials while allowing for good air flow, etc. The choice of a suitable container would be within the knowledge of the skilled artisan.

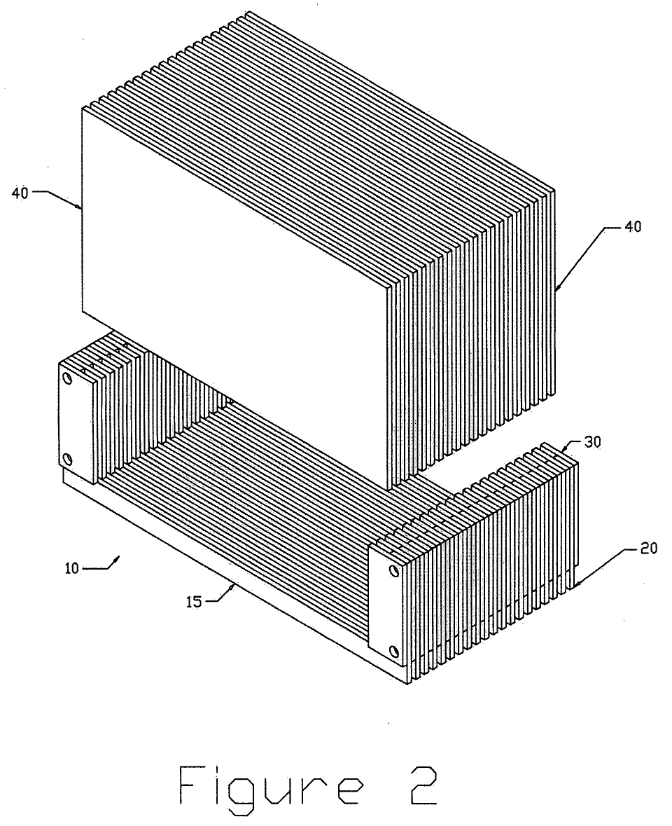

A preferred embodiment of the present invention is provided in FIGS. 1-2. Specifically, FIG. 1A shows base member 10 with horizontal bridge member 15 having two vertical side tabs 20, FIG. 1B shows spacer 30, and FIG. 1C shows base member 10 with two spacers 30 placed adjacent to vertical side tabs 20. When this assembly of FIG. 1C is paired with at least one other similar assembly to form a plurality of base members, as shown in the lower half of FIG. 2, the two spacers 30 between each of the multiple assemblies of FIG. 1C act to separate these assemblies.

Thus the lower half of one embodiment of the nematode harvesting system of the invention is shown in the lower half of FIG. 2, i.e., the portion of the harvesting system of the present invention which lacks the collection plate member or, in the case of FIG. 2, members. As discussed, this system of FIGS. 1-2 is composed of a plurality of base members 10, which in this particular embodiment are separated by spacers 30, as shown in detail in FIG. 1C. In use the apparatus of FIG. 2 is placed with vertical side tabs 20 pointing up, as shown in FIG. 2, and with the lower portion of horizontal bridge member 15 resting on, or partially embedded in, the semi-solid culture medium (not shown in FIG. 2) onto which the apparatus of FIG. 2 has been placed.

The upper half of the embodiment of the Figures is composed of a plurality of collection plate members 40, with an example of a single collection plate member provided in FIG. 1D. As FIG. 1D shows, the width of collection plate member 40 is slightly less than the distance between vertical side tabs 20 of base member 10 of FIG. 1A. Thus a collection plate member may be removably contacted against base member 10 by inserting it into the cavity formed by base member 10 flanked by four spacers 30, i.e., by the cavity formed when the base member/spacers assembly of Fig. 1C is flanked by another base member/spacer assembly, e.g., as is shown for the plurality of base members/spacers of FIG. 2.

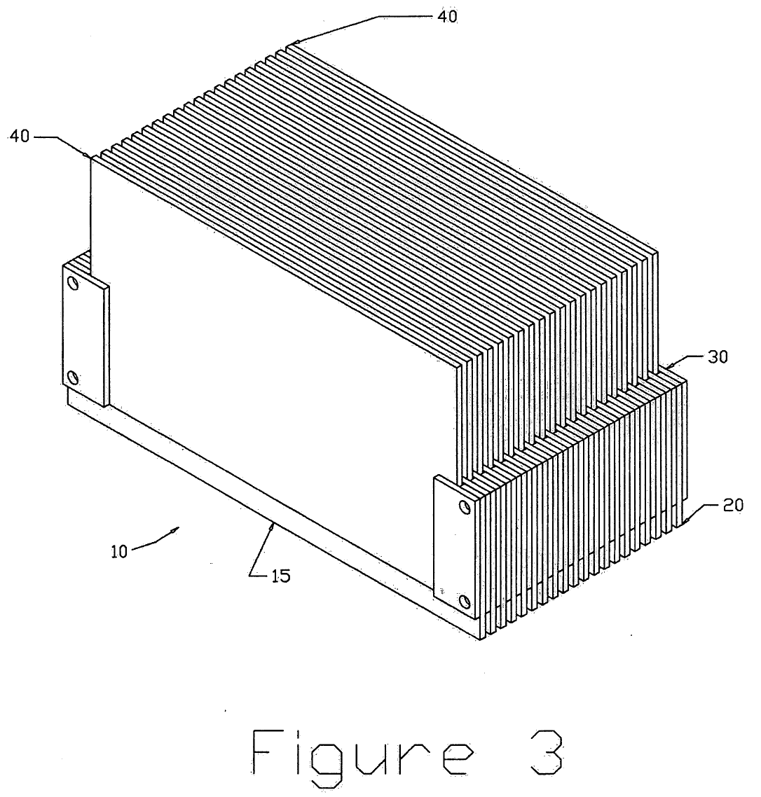

FIG. 3 shows an apparatus in which a plurality of collection plate members 40 have been removably contacted against a plurality of base members 10 separated by a plurality of spacers 30. In this Figure the collection plate members 40 are shown as being unconnected to one another, i.e., independently removable. In other embodiments of the invention means are provided for joining this plurality of collection plate members together into one unit, such that the entire plurality of collection plate members may be removed as a single unit from the plurality of base members. For example, the collection plate members 40 may be joined together by spacers, etc., or other means known to the skilled artisan.

### EXAMPLE 1

### Culture and Harvesting of Nematodes

Typically, oatmeal or cornmeal is mixed with water to form a moist, semi-solid (non-watery) paste, a small amount of yeast is added, and the resulting mixture is poured into a container and inoculated with nematodes.

The apparatus of the present invention may be contacted with the semi-solid culture medium at any time after the medium is poured, and in fact the present invention also explicitly contemplates that the medium may be poured into a container where the apparatus of the present invention has already been appropriately positioned. However, it is preferable that the apparatus of the invention is placed onto the culture-medium when the nematode population is sufficiently large that nematodes have begun to ascend the walls of the container in which they are growing. Although the apparatus may be introduced without further treatment, it will be advantageous to wet the collection plates, e.g., with a dousing or spraying of water, either before or after introduction of the apparatus into the container.

After the appropriate period during which nematodes migrate onto the collection plates, the collection plates (or plate, if the apparatus consists of a single pair of collection plate and base member) are withdrawn and the nematodes on the collection plate are removed by, e.g., dousing or spraying. The collection plates (or plate) may then be re-contacted with the base members (or base member) and the process repeated as desired.

While the present invention has been described with reference to its preferred embodiments, one of one of ordinary skill in the relevant art will understand that the present invention is not intended to be limited by these preferred embodiments, and is instead contemplated to include all embodiments consistent with the spirit and scope of the present invention as defined by the appended claims. The entire disclosures of all references, applications, patents, and publications cited herein are hereby incorporated by reference.

What is claimed is:

1. An apparatus for harvesting nematodes, wherein said apparatus comprises at least one paired set of collection plate member and corresponding base member, wherein said collection plate member is in removable contact with said base member.

2. The apparatus of claim 1, wherein said collection plate member is selected from the group consisting of a glass collection plate member, a plastic collection plate member, a metal collection plate member, a ceramic collection plate member, and a wood collection plate member.

3. The apparatus of claim 2, wherein said collection plate member is a plastic collection plate member.

4. The apparatus of claim 3, wherein said plastic collection plate member is a coated plastic collection plate member.

5. The apparatus of claim 1, wherein said apparatus comprises more than one paired set of collection plate member and corresponding base member.

6. The apparatus of claim 5, wherein said each paired set of collection plate member and corresponding base member is separated from the adjacent paired set of collection plate member and corresponding base member.

7. The apparatus of claim 6, wherein said each paired set of collection plate member and corresponding base member is separated from the adjacent paired set of collection plate member and corresponding base member by a spacer.

8. A method for harvesting nematodes, wherein said method comprises:
a) contacting the base member of the paired set of collection plate member and base member of the apparatus of claim 1 with nematode culture medium;
b) allowing nematodes to collect on the collection plate member of the paired set of collection plate member and base member of the apparatus of claim 1; and,
c) removing the collection plate member of the paired set of collection plate member and base member of the apparatus of claim 1 for nematode harvesting.

9. An apparatus for harvesting nematodes, wherein said apparatus comprises more than one collection plate member.
